(19) United States
(12) Patent Application Publication
(10) Pub. No.: US 2023/0025866 A1
(43) Pub. Date: Jan. 26, 2023

(54) NOVEL MANUFACTURING PROCESS FOR GADOLINIUM COMPLEXES

(71) Applicant: GE Healthcare AS, Oslo (NO)

(72) Inventors: Mikkel Jacob Thaning, Oslo (NO); Andreas Richard Meijer, Oslo (NO)

(21) Appl. No.: 17/787,545

(22) PCT Filed: Dec. 18, 2020
(86) PCT No.: PCT/EP2020/087323

§ 371 Date: Jun. 20, 2022

(30) Foreign Application Priority Data
Dec. 20, 2019 (GB) 1919073.5

(51) Int. Cl. A61K 49/10

(52) U.S. Cl. CPC A61K 49/108

(57) ABSTRACT

The present invention provides a novel method for removal of gadolinium ions from a solution comprising gadolinium complexed with DOTA. The method of the invention is relatively straightforward and cost-efficient compared with known methods.

### TECHNICAL FIELD OF THE INVENTION

The present invention relates generally to a method for removal of gadolinium ion from a complex of gadolinium with DOTA. The method uses active carbon to remove excess gadolinium ions. Also provided is a method of preparation of a Gd-DOTA magnetic resonance imaging (MRI) contrast agent comprising the inventive method of gadolinium removal.

### DESCRIPTION OF RELATED ART

Metal complexes of lanthanide metals, especially gadolinium, are of interest as MRI contrast agents in the field of in vivo medical imaging. MRI contrast agents based on metal complexes of gadolinium have been reviewed extensively [see e.g. Zhang et al, Curr. Med. Chem., 12, 751-778 (2005) and Aime et al, Adv. Inorg. Chem., 57, 173-237 (2005)].

Free gadolinium ions can, however, exhibit significant toxicity in vivo. U.S. Pat. No. 5,876,695 addresses this problem by including in the formulation of the gadolinium metal complex an additive, which is a ‘weak metal chelate complex’ such as with calcium. The idea is that the excess ‘weak metal chelate complex’ will complex efficiently any gadolinium ions which may adventitiously be either liberated or present, and thus improve the safety of the MRI contrast composition.

Reference Example 3 of EP 2242515 B9 includes a laboratory scale preparation which prepares Gd-DOTA by reaction of DOTA (10 g, 25 mmol) with a stoichiometric amount of gadolinium oxide (Gd₂O₃, 12.5 mmol) at 80° C. in water at pH 6 to 7 maintained with NaOH. The pH is then adjusted with HCl to 5, and residual free gadolinium removed by stirring with a Chelex resin in sodium ion form for 2-hours, followed by filtration. EP 2242515 B9 teaches that the Gd-DOTA complex is then precipitated from aqueous ethanol giving an 80% isolated yield of sodium gadoterate as a white powder. EP 2242515 B9 does not teach how the method of Reference Example 3 can be adapted to provide the liquid pharmaceutical composition having an excess of macrocyclic chelator in the range 0.002% and 0.4% mol/mol, in particular on an industrial scale.

Furthermore, the use of Chelex resin as taught by Example 3 of EP 2242515 B9 provides the product in sodium salt form unless further purification steps are carried out. Example 3 of EP 2242515 B9 also describes the preparation of a specific gadolinium complex which necessitates purification and isolation steps unsuitable for an industrial manufacturing process of preparation of a liquid pharmaceutical formulation.

WO 2016/083597 discloses a process for preparation of a liquid pharmaceutical formulation comprising a metal complex of a lanthanide metal with a macrocyclic chelator which includes a step of removal of the excess lanthanide by contacting one or more times with a scavenger resin, whereby the excess lanthanide is complexed to said scavenger resin

Unlike the experimental example of EP 2242515 B9, the method described in WO 2016/083597 can be carried out on an industrial scale. It avoids the need for measurement and adjustment steps as the lanthanide chelator metal complex is obtained without excess lanthanide ions being present by using a solid-phase bound scavenger chelator. Since this process provides an intermediate solution of the lanthanide metal complex without free lanthanide ions, the amount of excess macrocyclic chelator to add to give the desired formulation having a defined excess of free chelator can be calculated readily.

Nevertheless, there is still a need for alternative methods of removing excess lanthanide metals from formulations of lanthanide metal complexes of macrocyclic chelators. The methods should preferably be suitable for pharmaceutical manufacture on an industrial scale, and also be suitable for the provision of MRI contrast agents comprising such formulations.

### SUMMARY OF THE INVENTION

In one aspect the present invention relates to a method comprising the following steps:
(i) complexation of a chelate with an excess of gadolinium in a suitable solvent, to give a first solution comprising Gd-chelate and free gadolinium ions ([Gd_{free}]);
(ii) removal of [Gd_{free}] from the first solution of step (i) by contacting said solution one or more times with an amount of active carbon;
(iii) separation of the active carbon from the first solution of step (ii), to give a second solution which comprises said Gd-chelate free from excess [Gd_{free}].

In another aspect the present invention relates to a method comprising the following steps:
(A) carrying out method steps (i)-(iii) as defined herein to give the second solution as defined herein;
(B) addition of chelate in uncomplexed form to said second solution from step (A) to give a liquid pharmaceutical formulation comprising Gd-chelate, together with chelate in uncomplexed form.

In another aspect the present invention provides a method of preparation of an MRI contrast agent which comprises:
(a) carrying out the method steps (A) and (B) as defined herein to obtain the liquid pharmaceutical formulation as defined herein;
(b) optionally diluting the liquid pharmaceutical formulation from step (a) with a biocompatible carrier;
(c) dispensing the formulation from step (b) into pharmaceutically acceptable containers or syringes to give dispensed containers or syringes;
(d) either carrying out steps (a)-(c) under aseptic manufacturing conditions, or terminal sterilisation of the dispensed containers or syringes from step (c), to give the MRI contrast agent in said pharmaceutically acceptable containers or syringes in a form suitable for mammalian administration.

In another aspect the present invention provides a solution of Gd-chelate free from excess [Gd_{free}] obtainable by a method comprising steps (i)-(iii) as defined herein.

In another aspect the present invention provides a liquid pharmaceutical formulation comprising Gd-chelate, together with chelate in uncomplexed form obtainable according to the method comprising steps (A) and (B) as define herein.

In another aspect the present invention provides an MRI contrast agent obtainable according to the method comprising steps (a)-(d) as defined herein.

It has been demonstrated herein that active carbon is capable of the efficient removal of free gadolinium ions from a Gd-DOTA meglumine solution. The method of the present invention represents a relatively inexpensive and uncomplicated method for the production of Gd-chelates as compared with known methods.

It is demonstrated herein that active carbon treatment is an efficient method for removal of gadolinium ions in a gadoteric acid meglumine solution at a concentration and pH range that is representative of gadoterate meglumine manufacturing conditions.

The method of the present invention furthermore represents a significant simplification of previous methods (e.g., see WO 2016/083597) as the preconditioning steps of the scavenger resin are not required.

### BRIEF DESCRIPTION OF THE FIGURES

FIG. 1 shows the distribution of Gd³⁺ hydrolytic species in 0.1M LiCl at 298K. c_{Gd}=1.00 mM. (from Djurdjevic et al. Acta. Chim. Slov. 2010, 386-397)

FIG. 2 depicts gadolinium concentration [Gd] in spiked 0.5M Gd-DOTA solutions after titration.

FIG. 3 illustrates the effect of active carbon treatment. The magnified graph is included for clarity on low gadolinium concentration [Gd] samples.

FIG. 4 shows CAD chromatograms of selected active carbon treated samples. *DOTA elutes as copper complex.

FIG. 5 shows the absorbance ratio vs. gadolinium concentration [Gd] for a representative calibration curve.

### DETAILED DESCRIPTION OF THE PREFERRED EMBODIMENTS

To more clearly and concisely describe and point out the subject matter of the claimed invention, definitions are provided hereinbelow for specific terms used throughout the present specification and claims. Any exemplification of specific terms herein should be considered as a non-limiting example.

The terms “comprising” or “comprises” have their conventional meaning throughout this application and imply that the method must have the essential features or components listed, but that others may be present in addition. The term ‘comprising’ includes as a preferred subset “consisting essentially of” which means that the method has the steps listed without other features or steps being present.

For use in MRI, paramagnetic metal ions such as gadolinium are administered as metal chelates in order to avoid any toxic effects of these metal ions in their free form. As well as the paramagnetic metal ion being stably complexed, the geometry of the chelate should be such that the paramagnetic effectiveness of the metal ion is maintained. A “chelate” (also the term “cheland” is used to define the chelate without the metal) in the context of the present invention is any ligand capable of producing a highly stable metal chelate complex, e.g. one with a thermodynamic stability constant of at least 10¹². In various embodiments the chelate can be a linear, cyclic or branched chelating agent, e.g. a linear mono- or polychelant, a macrocydic chelant or a branched polychelant (e.g. a dendrimeric polychelant). In one embodiment the chelate will be a polyaminopolyoxyacid (e.g. polyaminopolycarboxylic acid).

In one embodiment of the invention the chelate is selected from the group comprising the following (or derivatives thereof): diethylenetriaminepentaacetic acid (DTPA); 4-carboxy-5, 8, 11-tris(carboxymethyl)-1-phenyl-2oxa-5, 8, 11-triazatridecan-13-oic acid (BOPTA); 1, 4, 7, 10-tetraazacyclododecan-1, 4, 7-triactetic acid (DO3A); 1, 4, 7, 10-tetraazacydododecan-1, 4, 7, 10-tetraactetic acid (DOTA); ethylenediaminotetraacetic acid (EDTA); 10-(2-hydroxypropyl)-1, 4, 7, 10-tetraazacydododecan-1, 4, 7-triacetic acid (HP-DO3A); 2-methyl-1, 4, 7, 10-tetraazacyclododecan-1, 4, 7, 10-tetraacetic acid (MCTA); tetramethyl-1, 4, 7, 10-tetraazacydododecan-1, 4, 7, 10-tetraacetic acid (DOTMA); 3, 6, 9, 15-tetraazabicyclo[9.3.1]pentadeca-1(15),11, 13-triene-3, 6, 9-triacetic acid (PCTA); N, N′Bis(2-aminoethyl)-1,2-ethanediamine (TETA); 1,4,7,10-tetraazacyclotridecane-N,N′,N″,N′″-tetraacetic acid (TRITA); 1,12-dicarbonyl, 15-(4-isothiocyanatobenzyl) 1, 4, 7, 10, 13-pentaazacyclohexadecane-N, N′, N″ triaceticacid (HETA); [(2S,5S,8S,11S)-4,7-bis-carboxymethyl-2,5,8,11-tetramethyl-1,4,7,10-tetraazacyclo-dodecan-1-yl]acetic acid, (M4DO3A); 1-O-Phosphonomethyl-1,4,7, 1-O-tetraazacyclododecane-1,4,7-triacetic acid (MPDO3A); hydroxybenzyl-ethylenediamine-diacetic acid (HBED); N,N′-ethylenebis-[2-(o-hydroxyphenolic)glycine](EHPG); 10-[(1SR,2RS)-2,3-dihydroxy-1-hydroxymethylpropyl]-1,4,7,10-tetraazacyclododecane-1,4,7-triacetic acid (BT-DO3A); and, 2-[bis[2-[carboxylatomethyl-[2-(2-methoxyethylamino)-2-oxoethyl]amino]ethyl]amino]acetate (DTPA-BMEA).

In one embodiment the chelate is selected from DTPA, DOTA or derivatives thereof. In a yet further embodiment said chelate or derivative thereof is selected from EOB-DTPA, DTPA-BMA, DTPA-BMEA, DTPA, DOTA, BOPTA, HP-DO3A and BT-DO3A. In one embodiment the chelate is DOTA.

The terms “chelate in uncomplexed form” and chelate “free of coordinated gadolinium ions” and “free chelate” refers to any of the above-described chelates of the invention with no gadolinium co-ordinated thereto. For example DOTA in uncomplexed form has the following structure:

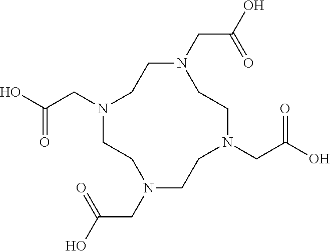 [SMILES: O=C(O)CN1CCN(CC(=O)O)CCN(CC(=O)O)CCN(CC(=O)O)CC1]

A “complex of gadolinium with chelate” refers the chelate including the co-ordinated metal. For example, a complex of gadolinium with DOTA (or “Gd-DOTA chelate” and also referred to herein as Gd-DOTA or gadoterate) refers to the following:

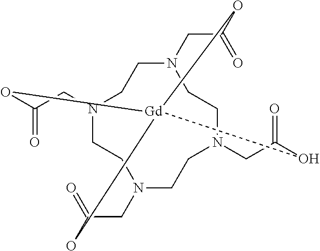

The “meglumine salt of Gd-DOTA” or “meglumine salt of Gd-DOTA chelate” refers to the following:

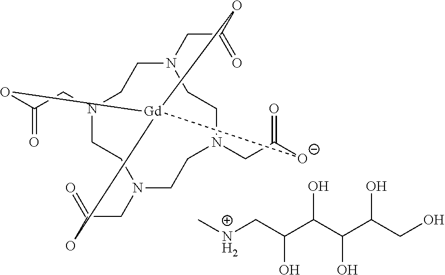

The term “active carbon” (also commonly referred to as activated carbon, charcoal, activated powder, carbon black, Carboraffin, Carborafine) refers to any active carbon known in the art including active carbon as a form of carbon processed to have small, low-volume pores to provide a large surface area.

The term as used herein also encompasses surface-modified forms of active carbon. Active carbon may be provided as particles, pellets or as a mesh all of which are readily available commercially. One non-limiting example of commercially-available active carbon is active carbon mesh 100 from Sigma Aldrich (161551-175-D). Active carbon is commonly used on a laboratory scale to purify solutions of organic molecules containing unwanted coloured organic impurities. Filtration over activated carbon is well-known in large scale fine chemical and pharmaceutical processes for the same purpose.

The term “purification” as used herein refers to the process(es) to obtain a substantially gadolinium ion-free version of the desired product, i.e. Gd-chelate with [Gd_{free}] removed. The term “substantially” refers to the complete or nearly complete extent or degree of an action, characteristic, property, state, structure, item, or result. The term “substantially pure” herein encompasses Gd-chelate with zero [Gd_{free}] as well as Gd-chelate where only minute quantities of [Gd_{free}] remain such that any subsequent steps can be successfully carried out. The phrase “free from excess [Gd_{free}]” herein can be understood to be synonymous with “substantially pure”.

The term “free gadolinium ions” or “[Gd_{free}]” refers to Gd³⁺ in solution that is not complexed with chelate.

The term “complexation” herein refers to the process by which a metal ion (here gadolinium ion) is bound through multiple ligands of a chelating agent.

The term “suitable solvent” refers to any solvent or solvent system in which the complexation of chelate with gadolinium can take place. Water is an example of a suitable solvent, with water for injection (WFI) being particularly suitable.

The term “contacting” in the context of contacting a reaction solution with active carbon can be taken to mean addition of the solution to the active carbon or addition of active carbon to the solution. In one embodiment addition of the solution to the active carbon is carried out by passing the solution through a column or cartridge containing the active carbon, optionally where the flow of solution through the column is facilitated by use of a pump.

The phrase “one or more times” can encompass one or several contact steps as required to reduce the concentration of gadolinium ions to the desired level. Ideally fewer times is better.

The term “separation” refers to the physical removal of the active carbon from the solution. In one embodiment separation is carried out by filtration. In certain embodiments separation is carried out after each contacting step. Where the active carbon is contained in a column separation is automatically achieved as the solution passed out of the column. Nevertheless, where active carbon is contained in a column, the method of the invention may include a separate separation step whereby any fine particles of active carbon that may be present are removed. A “pharmaceutical formulation” can be understood to be a composition of Gd-chelate or a salt or solvate thereof together with a biocompatible carrier in a form suitable for mammalian administration. The “biocompatible carrier” is a fluid, especially a liquid, in which Gd-chelate is dissolved, such that the resulting composition is physiologically tolerable, i.e. can be administered to the mammalian body without toxicity or undue discomfort. By the phrase “in a form suitable for mammalian administration” is meant a composition which is sterile, pyrogen-free, lacks compounds which produce toxic or adverse effects, and is formulated at a biocompatible pH. Such compositions lack particulates which could risk causing emboli in vivo, and are formulated so that precipitation does not occur on contact with biological fluids (e.g. blood). Such compositions also contain only biologically compatible excipients, and are preferably isotonic.

The pharmaceutical formulation is suitable for use as an “MRI contrast agent”, i.e. to carry out MRI of the human and non-human animal body. The pharmaceutical formulation comprises one or more pharmaceutically-acceptable excipients. These suitably do not interfere with the manufacture, storage or use of the final composition. Non-limiting examples of suitable pharmaceutically-acceptable excipients include buffering agents, stabilizers, antioxidants, osmolality adjusting agents, pH adjusting agents, excess free chelate and weak complexes of physiologically tolerable ions. These and other suitable excipients will be well known to those of skill in the art and are further described in e.g. WO1990003804, EP0463644-A, EP0258616-A and U.S. Pat. No. 5,876,695 the content of which are incorporated herein by reference. The pharmaceutical formulation of the invention in one embodiment is in a form suitable for parenteral administration, for example injection. The pharmaceutical formulation may therefore be formulated for administration using physiologically acceptable excipients in a manner fully within the skill of the art. For example, Gd-chelate, optionally with the addition of pharmaceutically acceptable excipients, may be suspended or dissolved in an aqueous medium, with the resulting solution or suspension then being sterilized.

A non-limiting example of a suitable buffering agent is tromethamine hydrochloride.

The term “excess free chelate” (also can be referred to as “excess cheland” or “free ligand”) is defined as any compound capable of scavenging free gadolinium ion. The presence of excess free chelate ensures that no free gadolinium will be formed during the certified shelf life of the product. Degradation of a gadolinium-containing ligand can in principle lead to free gadolinium and excess free chelate will complex liberated gadolinium ions and ensure zero concentration of free Gd. Furthermore, it is known that there is a correlation between the amount of excess free chelate in a paramagnetic chelate formulation and the amount of paramagnetic metal deposited in animal models (Sieber 2008 J Mag Res Imaging; 27(5): 955-62). The amount of excess free chelate is selected that can act as a gadolinium scavenger to reduce or prevent release of gadolinium during its shelf life and from the formulation in vivo post injection. The optimal amount of free chelate will result in a pharmaceutical formulation having suitable physicochemical properties (i.e. viscosity, solubility and osmolality) and avoiding toxological effects such as zinc depletion in the case of too much free chelate. In one embodiment the excess free chelate is as defined hereinabove for “chelate in uncomplexed form”. In one embodiment of the present invention excess free chelate is DOTA in uncomplexed form. Where DOTA is used as excess free chelate in one embodiment it is present in the range 0.002 and 0.4 mol/mol %.

A “physiologically tolerable ion” may in one embodiment be selected from calcium salts or sodium salts such as, for example, calcium chloride, calcium ascorbate, calcium gluconate or calcium lactate.

Parenterally administrable forms should be sterile and free from physiologically unacceptable agents and should have low osmolality to minimize irritation or other adverse effects upon administration and thus the pharmaceutical composition should be isotonic or slightly hypertonic. Non-limiting examples of suitable vehicles include aqueous vehicles customarily used for administering parenteral solutions such as Sodium Chloride Injection, Ringers Injection, Dextrose Injection, Dextrose and Sodium Chloride Injection, Lactated Ringers Injection and other solutions such as are described in Remington's Pharmaceutical Sciences, 22^{nd} Edition (2006 Lippincott Williams & Wilkins) and The National Formulary (https://books.google.com/books?id=O3qixPEMwssC&q=THE+NATIONAL+FORMULARY&dq=THE+NATIONAL+FORMULARY&hl=en&sa=X&ved=0CC8Q6 AEwAGoVChMImfPHrdTqyAIVJfNyCh1RJw_E).

For the pharmaceutical composition of the invention to be administered parenterally, i.e. by injection its preparation further comprises steps including removal of organic solvent, addition of a biocompatible buffer and any optional further ingredients such as excipients or buffers. For parenteral administration, steps to ensure that the pharmaceutical composition is sterile and apyrogenic also need to be taken. Sterility can be achieved using “aseptic manufacturing conditions”, i.e. where sterility is maintained throughout the manufacturing process. Alternatively, “terminal sterilisation” can be used, i.e. where a final step assuring sterility of the product is carried out. “Pharmaceutically acceptable containers or syringes” suitably assure patient safety, the efficacy of the pharmaceutical formulation through the intended shelf life, uniformity of the pharmaceutical formulation through different production lots, control of possible migration of packaging components into the pharmaceutical formulation, control of degradation of the pharmaceutical formulation by oxygen, moisture, heat, etc., prevention of microbial contamination, sterility, etc. Suitable containers and syringes can be made from pharmaceutically acceptable glass or plastic. An exemplary plastic bottle is the Pluspak™.

The term “ambient temperature” herein can be taken to mean any temperature between about 18-30° C.

In one embodiment the target [Gd_{free}] is in the range 0.1-0.9 mM, in one embodiment 0.2-0.85 mM, in another embodiment 0.32-0.83 mM. In one embodiment the concentration of free gadolinium ions in the product following contact with active carbon endpoint is not more than 1.8 μg/mL.

In one embodiment said Gd-chelate is selected from; gadoterate (Dotarem), gadodiamide (Omniscan), gadobenate (MultiHance), gadopentetate (Magnevist), gadoteridol (ProHance), gadofosveset (Ablavar, formerly Vasovist), gadoversetamide (OptiMARK), gadoxetate (Eovist or Primovist) and gadobutrol (Gadavist).

In one embodiment said chelate is a macrocyclic chelate. In one embodiment said macrocyclic chelate is DOTA. In one embodiment said Gd-chelate is gadoterate. In one embodiment said Gd-chelate is gadoterate meglumine.

In one embodiment the excess gadolinium of step (i) is 0.001 to 5 mol/mol %.

In one embodiment following complexation step (i) and before removal step (ii) the pH is adjusted to 4.5 to 7.0, for example 4.5 to 6.5.

In one embodiment said Gd-chelate is gadoterate meglumine and said pH is adjusted using meglumine.

In one embodiment said amount of active carbon per millilitre of first solution is 200-400 mg/ml, in one embodiment 100-200 mg/ml and in another embodiment 10-20 mg/ml.

In one embodiment said active carbon is in the form of powder or particles, or alternatively it may be shaped or incorporated into other forms, e.g. pellets, discs or a mesh. Where active carbon is shaped into other forms, it may be held together by a binding agent, e.g. cellulose.

In one embodiment said active carbon is in the form of particles.

In one embodiment said active carbon is packed into a column or cartridge. In this embodiment the active carbon may be in any of the forms described herein. Optionally, the active carbon is supported by a scaffold within the column, which can be useful for active carbon shaped or incorporated into forms as described above.

In one embodiment where the active carbon is contained in a column, the column is a housing wherein active carbon is present as one or more stationary discs made from active carbon held together by a cellulose binding agent.

In one embodiment step (ii) is carried out at ambient temperature.

For preparation of a liquid pharmaceutical formulation comprising Gd-chelate, together with chelate in uncomplexed form according to an aspect of the present invention said Gd-chelate is Gd-DOTA and said chelate is DOTA and said DOTA in uncomplexed form is in an amount in the range 0.002 and 0.4 mol/mol % of said Gd-DOTA. In one embodiment the DOTA in uncomplexed form is in an amount in the range 0.025 and 0.25 mol/mol %.

In one embodiment the DOTA uncomplexed form is free of coordinated gadolinium ions and comprises less than 50 ppm M wherein M is a metal ion chosen from calcium, magnesium and zinc, or mixtures thereof.

As described in Example 2 below, to study the removal of free gadolinium ions, a meglumine Gd-DOTA liquid bulk solution was diluted to 0.5M (Gd-DOTA) using water, followed by addition of gadolinium ions (gadolinium chloride) to form a gadolinium spiked meglumine Gd-DOTA solution. The obtained solution was theoretically 0.5M (with respect to [Gd-DOTA]) and the concentration of excess free gadolinium ions was determined to be 1.4 mM, according to spectrophotometric analysis.

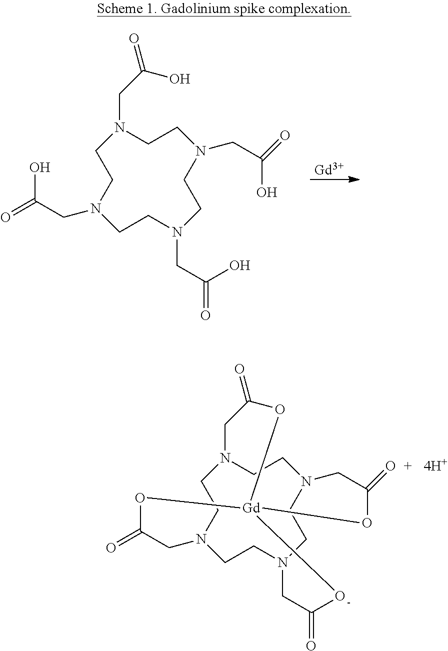

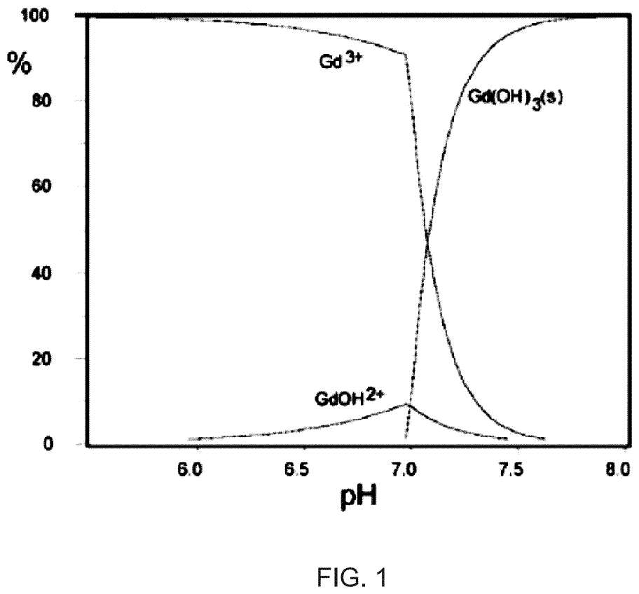

The pH drops from 7.3 to 4.8 during addition of gadolinium ions to the Gd-DOTA solution (Scheme 1 above). This drop in pH ensures that the spiked gadolinium ions indeed are in a free ionic form as it is known that gadolinium ions will form hydrolytic species with hydroxide ions at pHs from 6 and higher (FIG. 1), that eventually will form insoluble gadolinium hydroxide; Gd(OH)₃.

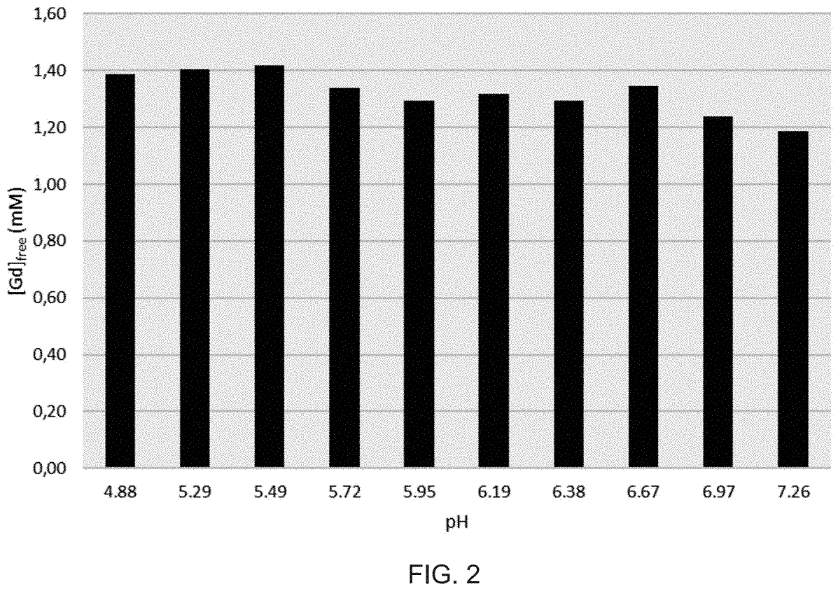

To ensure that no insoluble gadolinium hydroxide is formed in the spiked meglumine Gd-DOTA solutions during the experiments, the pH was titrated and aliquots were drawn at increasing pH and allowed to stand for >72 before analysis of free gadolinium content. As can be seen in FIG. 2, the free gadolinium concentration does not drop in the pH range relevant for active carbon experiments. Additionally, the gadolinium concentration [Gd] drops only slightly at a pH close to 7, indicating a lower tendency to form insoluble complexes in a 0.5M Gd-DOTA solution than in an aqueous solution (FIG. 1) as reported by Djurdevic et al (Acta. Chim. Slov. 2010, 386-397).

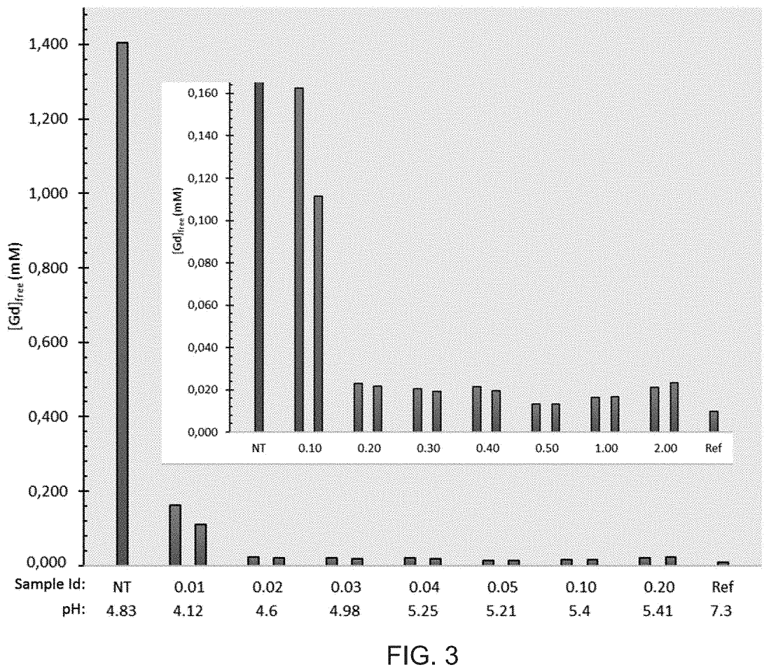

The abovementioned Gd spiked Gd-DOTA samples were subjected to active carbon treatment and the amount of remaining free Gd was determined using spectrophotometry. FIG. 3 compares the [Gd]_{free} of the spiked Gd-DOTA solution prior to active carbon treatment (NT; not treated) to solutions treated with 0.01-0.2 g/mL of active carbon, after 10 and 50 minutes of contacting time. The data indicate that as little as 0.02 g/mL of active carbon is capable of reducing the concentration of free gadolinium from 1.4 mM to roughly 0.02 mM. The 0.01 g/mL sample showed a lower [Gd] after 50 min compared to 10 min, it is not known if the system had reached equilibrium at this point or if further adsorption is possible. The rest of the samples (>0.01 g/mL) had reached equilibrium after the first time point, indicative of an excessive active carbon load.

As apparent in FIG. 3 it is not possible to further reduce the concentration of free gadolinium by adding more active carbon and it is possible that 0.02 mM is an overestimation due to background interference with the spectrophotometric method. A reference sample (Ref) of meglumine Gd-DOTA solution post dilution with water but prior to gadolinium addition, indicates that there is indeed some minor spectrophotometric background interference. Note that the reference solution contains a slight excess of DOTA and this possibly has some minor adverse effect on the background interference. It is therefore quite possible that the concentration of free gadolinium is lower than the 0.02-0.01 mM that the method indicates. Nevertheless, a gadolinium ion concentration of 0.01 mM is still acceptable for manufacturing gadoterate meglumine, as subsequent DOTA addition (in the 0.35-0.87 mM range) still would be considered to be practically quantitative.

As can be seen from FIG. 3, the pH is affected slightly by the active carbon treatment, however still well within acceptable ranges with respect to process requirements and solubility of gadolinium ions (FIG. 2).

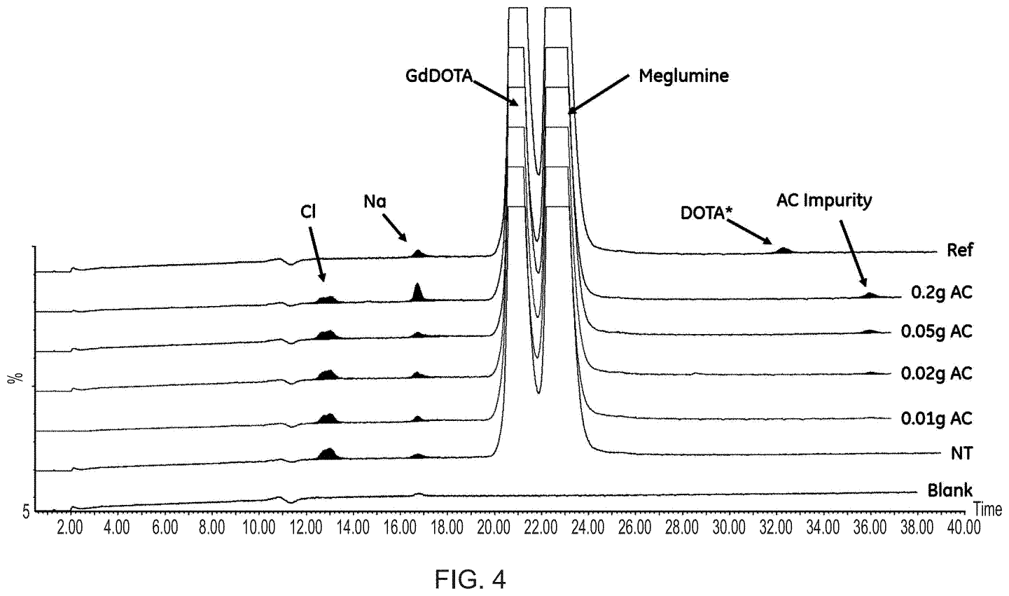

HPLC-CAD-MS analysis of active carbon treated samples indicate that no new compounds are formed due to degradation or chemical incompatibility (as illustrated in FIG. 4). Besides minor impurities introduced by the active carbon (small amounts of sodium and an unidentified impurity) or the spiking operation (chloride ions) no new peaks were found.

While the specific examples presented herein relate to meglumine Gd-DOTA these data support the application of the present invention to the effective removal of free gadolinium ions from a range of different solutions comprising a Gd-chelate.

This written description uses examples to disclose the invention, and also to enable any person skilled in the art to practice the invention, including performing any incorporated methods. The patentable scope of the invention is defined by the claims, and may include other examples that occur to those skilled in the art. Such other examples are intended to be within the scope of the claims if they have structural elements that do not differ from the literal language of the claims, or if they include equivalent structural elements with insubstantial differences from the literal languages of the claims. All patents and patent applications mentioned in the text are hereby incorporated by reference in their entireties, as if they were individually incorporated.

### BRIEF DESCRIPTION OF THE EXAMPLES

Example 1 is a comparative example describing a known method to remove free Gd ions from a meglumine Gd-DOTA solution.

Example 2 is a method of the invention for removal of free Gd ions from a meglumine Gd-DOTA solution.

List of Abbreviations used in the Examples

| | |
|---|---|
| CAD | charged aerosol detector |
| DAD | diode array detector |
| DIW | deionised water |
| HPLC | high performance liquid chromatography |
| MeCN | acetonitrile |
| OAc | acetate |
| RT | room temperature |
| TOF | time of flight |
| UPLC | ultra performance liquid chromatography |

### EXAMPLES

### Comparative Example 1: Removal of Gd Ions from a Meglumine Gd-DOTA Solution According to a Prior Art Method

DOTA (211 kg) was dissolved in boiling water (1600 kg) and Gd₂O₃ was added (94.8 kg). The temperature was set to 70° C. and the slurry was stirred over-night. The presence of free gadolinium ions (1390 ug/g) in the solution was determined by colorimetric titration.

The temperature was adjusted to 50° C. and meglumine was added to achieve pH 5.5 in the solution. Initially 94.8 kg meglumine was added and the final adjustment of the pH was made with an aqueous solution of meglumine (1.5 M).

Scavenger resin (Puropack C150, 50 L) was conditioned to proton form according to standard procedures. The resin was rinsed with water until neutral water was eluted from the resin bed. A solution of meglumine (400 g/kg resin) was cycled through the resin bed for 10 h and the resin was again rinsed to neutral pH with water.

The megluminized resin was placed in a column and the Gd-DOTA solution was pumped through the column at a flow rate sufficient to pass the entire volume of solution in 2 h. The concentration of free gadolinium (45 ug/ml) was determined using colorimetric spectrophotometry. The ion exchange of the meglumine Gd-DOTA solution was continued with one more passage through the column to establish a level of free gadolinium below detection limit by colorimetric titration (4 ug/g), to give a Gd-DOTA-meglumine solution.

### Example 2: Removal of Gd Ions from a Meglumine Gd-DOTA Solution Using a Method of the Present Invention

0.44M GdCl₃ solution was prepared from 1.86 g GdCl₃ hexahydrate dissolved in 10 mL deionized water. The concentration of gadolinium ([Gd]) was determined using a spectrophotometric method.

Gd spiked (1.4 mM) meglumine Gd-DOTA solution prepared from 50 mL of liquid bulk Clariscan™ meglumine Gd-DOTA to which was added 0.6 mL of GdCl₃ (0.44M) followed by dilution to 100 mL using DIW. The solution was then allowed to stand for >72 h before use in experiments to allow for complete complexation.

To 10 mL of Gd spiked (1.8 mM) meglumine Gd-DOTA solution was added 0.1, 0.2, 0.3, 0.4, 0.5, 1 or 2 g active carbon (mesh 100, Sigma Aldrich, 161551-175-D). Suspensions were placed in a shaking block and samples were taken after 10 and 50 minutes. All samples were filtered on Acrodisc nylon membranes and analyzed for [Gd] and HPLC purity (pH was measured in the suspension).

HPLC analysis was performed on an Agilent Acquity UPLC system equipped with a Diode Array Detector, a Waters Premier TOF mass spectrometer and a Dionex Charged Aerosol Detector.

Column: ZIC-pHILIC, 4.6×150 mm, 5 μm

Mobile phase: A: 100 mM NH4OAc; B: MeCN,

Flow: 1 mL/min

Gradient: 15% A to 30% A over 40 min (linear).

Temperature: (RT)

Detector: DAD (210-350 nm), CAD (500 pA)

Injection: Full loop injection (20 uL).

HPLC Sample preparation: To 380 μL water was added 10 μL sample followed by 10 μL Cu(OAc)2 (10 mg/ml) and 600 μL MeCN.

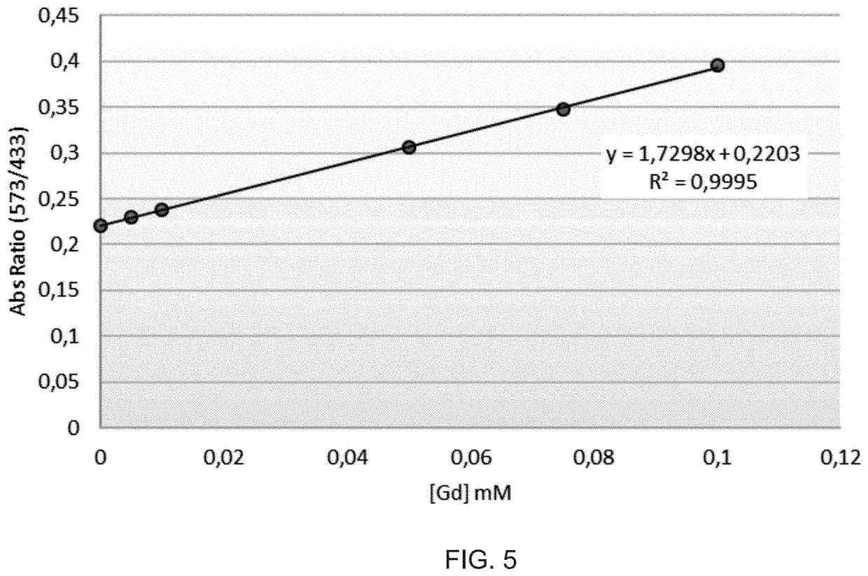

The concentration of free gadolinium was determined using a slightly modified published method (Barge, A; Contrast Media & Molecular Imaging, 2006, 184-8), based on the differences in the visible spectra of free and complexed Xylenol orange. The absorbance ratio of wavelengths 573 and 433 nm is proportional to the [Gd] in the 0 to 0.1 mM range (FIG. 5). At higher ranges, the absorbance ratio deviates from linearity and shows a more complex relationship with [Gd]. All measured samples were therefore diluted to fit the 0-0.1 mM calibration range.

GdCl₃ standard solutions were prepared from GdCl₃ hexahydrate and serial dilutions using 50 mM HOAc buffer.

50 mM HOAc was buffer prepared by adding 0.72 mL HOAc (conc.) to 200 mL deionized water, adjusting pH to 5.9 using 1M NaOH, then diluting to 250 mL using deionized water.

Xylenol Orange solution prepared by dissolving 2 mg Xylenol Orange in 100 mL 50 mM HOAc (pH 5.9) buffer.

Spectrophotometric sample preparation: To 25 μL of sample solution was added 975 μL Xylenol Orange solution.

### Example 3: Industrial Manufacture of Gd-DOTA Meglumine Using a Method of the Present Invention

DOTA (370 kg (range: 310-410 kg)) was dissolved in boiling water (2000 kg) and Gd₂O₃ (162.8 kg range: 135-190 kg)) was added. The temperature was set up to 80° C. and stirred over-night. The presence of free gadolinium ions was determined by colorimetric titration.

The temperature was adjusted to 50° C. and meglumine was added to achieve pH 5.7 (range 5.5-6.4). Initially 160 kg (range 150-200 kg) meglumine was added and the final adjustment of pH was made with an aqueous solution of meglumine (1.5M)

The temperature of solution Gd-DOTA meglumine was further adjusted to 40° C. 4 modules of MCN active carbon were placed in series. The first 3 filter houses contained 1.01 kg active carbon and the last one contained 2.7 kg of active carbon. MCN active carbon preconditioned before the Gd-DOTA meglumine solution was pumped. The MCN active carbon was rinsed with water until it reached conductivity below 10 ρS/cm. The GdDOTA solution was pumped through the 4 series of active carbon at a flow rate of 800-1000 liter/m²·hr. The concentration of free gadolinium was determined using calorimetric titration. The GdDOTA solution after passing the active carbon establish a level of free gadolinium below detection limit by colorimetric titration (1.8 μg/ml) to give a Gd-DOTA meglumine solution.

The data produced from the industrial manufacture is shown in the table below.

| | | | Initial | End concen- |
|---|---|---|---|---|
| | Concen- | Amount | concentration | tration free |
| | tration | Gd-DOTA | free Gadolinium | Gadolinium |
| | GD-DOTA | meglumine | before active | after active |
| | solution | solution | carbon | carbon |
| | [M] | [liter] | [μg/ml] | [μg/ml] |
| Example-1 | 0.46 | 179 | 41.9 | NMT 1.8 |
| Example-2 | 0.46 | 171 | 34.7 | NMT 1.8 |
| Example-3 | 0.41 | 164 | 34.2 | NMT 1.8 |

1) A method comprising the following steps:
(i) complexation of a chelate with an excess of gadolinium in a suitable solvent, to give a first solution comprising Gd-chelate and free gadolinium ions ([Gd_{free}]);
(ii) removal of [Gd_{free}] from the first solution of step (i) by contacting said solution one or more times with an amount of active carbon;
(iii) separation of the active carbon from the first solution of step (ii), to give a second solution which comprises said Gd-chelate free from excess [Gd_{free}].

2) The method as defined in claim 1 wherein target [Gd_{free}] is in the range 0.1-0.9 mM.

3) The method as defined in claim 1 wherein said Gd-chelate is selected from gadoterate, gadodiamide, gadobenate, gadopentetate, gadoteridol, gadofosveset, gadoversetamide, gadoxetate or gadobutrol.

4) The method as defined in claim 1 wherein said chelate is a macrocyclic chelate.

5) The method as defined in claim 1 wherein said macrocyclic chelate is DOTA.

6) The method as defined in claim 1 wherein said Gd-chelate is gadoterate.

7) The method as defined in claim 6 where said Gd-chelate is gadoterate meglumine.

8) The method as defined in claim 1, wherein the excess gadolinium of step (i) is 0.001 to 5 mol/mol %.

9) The method of claim 1, where following complexation step (i) and before removal step (ii) the pH is adjusted to 4.5 to 7.0.

10) The method as defined in claim 9 wherein said Gd-chelate is gadoterate meglumine and said pH is adjusted using meglumine.

11) The method as defined in claim 1 wherein said amount of active carbon relative to said first solution is 200-400 mg/ml.

12) The method as defined in claim 1 wherein said active carbon is in the form of particles, pellets or a mesh.

13) The method as defined in claim 1 wherein said active carbon is in the form of particles.

14) The method as defined in claim 1 wherein said active carbon is packed into a column or cartridge.

15) The method as defined in claim 1 wherein step (ii) is carried out at ambient temperature.

16) A method comprising the following steps:
(A) carrying out the method of claim 1 to give the second solution as defined therein;
(B) addition of chelate in uncomplexed form to said second solution from step (A) to give a liquid pharmaceutical formulation comprising Gd-chelate, together with chelate in uncomplexed form.

17) The method as defined in claim 16 wherein said Gd-chelate is Gd-DOTA and said chelate is DOTA and said DOTA in uncomplexed form is in an amount in the range 0.002 and 0.4 mol/mol % of said Gd-DOTA.

18) The method as defined in claim 17, where the DOTA in uncomplexed form is in an amount in the range 0.025 and 0.25 mol/mol %.

19) The method as defined in claim 17, where the DOTA uncomplexed form is free of coordinated gadolinium ions and comprises less than 50 ppm M wherein M is a metal ion chosen from calcium, magnesium and zinc, or mixtures thereof.

20) A method of preparation of an MRI contrast agent which comprises:
(a) carrying out the method as defined in claim 16 to obtain the liquid pharmaceutical formulation as defined therein;
(b) optionally diluting the liquid pharmaceutical formulation from step (a) with a biocompatible carrier;
(c) dispensing the formulation from step (b) into pharmaceutically acceptable containers or syringes to give dispensed containers or syringes;
(d) either carrying out steps (a)-(c) under aseptic manufacturing conditions, or terminal sterilisation of the dispensed containers or syringes from step (c), to give the MRI contrast agent in said pharmaceutically acceptable containers or syringes in a form suitable for mammalian administration.

21) The method of claim 20, where terminal sterilisation is used.

22) A solution of Gd-chelate free from excess [Gd_{free}] obtainable by the method as defined in claim 1.

23) A liquid pharmaceutical formulation comprising Gd-chelate, together with chelate in uncomplexed form obtainable according to the method as defined in claim 16.

24) An MRI contrast agent obtainable according to the method as defined in claim 20.
